(19) United States
(12) Patent Application Publication
(10) Pub. No.: US 2025/0034070 A1
(43) Pub. Date: Jan. 30, 2025

(54) METHOD AND SYSTEM FOR PREPARING DIMETHYL ETHER

(71) Applicant: BASF SE, Ludwigshafen am Rhein (DE)

(72) Inventors: Florian WINKLER, Pullach (DE); Virginie LANVER, Ludwigshafen am Rhein (DE); Nils BOTTKE, Ludwigshafen am Rhein (DE); Manuel Frederik GENTZEN, Ludwigshafen am Rhein (DE); Silvia TAURO, Ludwigshafen am Rhein (DE)

(21) Appl. No.: 18/715,783

(22) PCT Filed: Dec. 6, 2022
(86) PCT No.: PCT/EP2022/025553

§ 371 Date: Jun. 3, 2024

(30) Foreign Application Priority Data
Dec. 7, 2021 (EP) 21020619.9

(51) Int. Cl. C07C 41/09; B01J 8/06; C07C 29/154

(52) U.S. Cl. CPC C07C 41/09; B01J 8/065; B01J 8/067; C07C 29/154; B01J 2208/025; B01J 2208/065

(57) ABSTRACT

A process for synthesis of dimethyl ether is proposed, in which a feed gas mixture (E) containing carbon dioxide and/or carbon monoxide and hydrogen is guided through reaction tubes (1) of a cooled shell and tube reactor (10) that are equipped with a first catalyst and a second catalyst, in which the carbon dioxide present in the feed gas mixture (E) and/or the carbon monoxide present in the feed gas mixture is at least partly reacted over the first catalyst with the hydrogen present in the feed gas mixture to give methanol, and in which, in the same shell and tube reactor (10), the methanol is at least partly converted over the second catalyst to dimethyl ether. It is envisaged that the reaction tubes (1) are equipped with the first and second catalysts in the form of a structured bed, where the structured bed has two to four regions (11, 12, 13) in which the first and second catalysts are provided in different physical mixtures, where the regions (11, 12, 13) are arranged such that the concentration of the first catalyst increases in a flow direction through the reaction tubes (1). Also proposed is a plant (100) for implementation of such a process.

The invention relates to a process and to a plant for preparation of dimethyl ether from synthesis gas according to the preamble of the independent claims.

### BACKGROUND OF THE INVENTION

Dimethyl ether (DME) is the simplest ether in structural terms. It contains two methyl groups as organic radicals, is polar, and finds use for different purposes in industry.

Dimethyl ether can be prepared by a two-stage synthesis from synthesis gas via the intermediate methanol, as described, for example, in chapter 4 of the DME Handbook, Japan DME Forum, Tokyo 2007, ISBN 978-4-9903839-0-9. It is a feature of a “two-stage” synthesis that methanol is first prepared from synthesis gas, where the methanol is separated from unconverted synthesis gas and the methanol is then dehydrated separately and in a further step to dimethyl ether and water.

In a one-stage synthesis of dimethyl ether, by contrast, all reactions proceed in one and the same reactor and in one and the same catalyst bed. The term “direct synthesis” is also used hereinafter for a one-stage synthesis of dimethyl ether. The reactions that proceed are a reaction of hydrogen with carbon monoxide and/or carbon dioxide to give methanol and a (further) reaction of methanol to give dimethyl ether and water. The one-stage synthesis of dimethyl ether is known, for example, from U.S. Pat. Nos. 4,536,485 A and 5,189,203 A. It is conventional here to use hybrid catalysts. The reaction is exothermic and typically proceeds at a temperature of 200 to 300° C. and at a pressure of 20 to 100 bar.

Especially in relation to the terminology used hereinafter, but also to fundamental considerations relating to the synthesis of dimethyl ether from synthesis gas, reference is made to the article by I. Kiendl et al., Chem. Ing. Tech. 2020, 92, No. 6, 736-745. The terms are used here correspondingly.

The synthesis gas required for the preparation of dimethyl ether can be provided by a multitude of suitable technologies, as described, for example, in Hiller et al. in the article “Gas Production” in Ullmann's Encyclopedia of Industrial Chemistry, online edition, 15 Dec. 2006, doi 10.1002/14356007.a12_169.pub2, and from a multitude of starting materials.

In the two-stage synthesis of dimethyl ether, for the conversion to methanol, it is possible to use copper catalysts (also referred to hereinafter as “methanol catalysts”) and, for the subsequent step of dehydration, acidic catalysts in particular, such as zeolite catalysts, catalysts based on γ-dialuminum trioxide and aluminosilicate catalysts (also referred to hereinafter as “dehydration catalysts”). The synthesis gas for generation of methanol is notable for a stoichiometry value (for a definition, reference is made explicitly to Kiendl et al.) of somewhat more than 2.

The two-stage dimethyl ether synthesis produces dimethyl ether with high purity. The methanol from the first stage is freed of higher alcohols and then dehydrated in a highly selective manner. In a one-stage process, by-products are a further challenge, since methanol synthesis and dimethyl ether synthesis proceed in parallel here, and the higher alcohols formed are converted to olefins and occur in the product depending on the boiling situation.

One-stage dimethyl ether synthesis has not yet been achieved industrially. This is in particular because of the difficulty of integrating the two catalysts in one reactor. Different temperature and partial pressure conditions exist for the reactants and products in the integrated reactor than in the individual reactors, which makes it significantly more difficult to control the reaction temperature and accelerates aging of the catalyst, for example as a result of elevated concentrations or partial pressures of the by-products. Means of temperature control in the direct synthesis of dimethyl ether are described in WO 2019/122078 A1.

Because of the higher temperatures and elevated partial hydrogen pressures by comparison with the conventional methanol synthesis, copper catalysts are more likely to undergo sintering under these conditions. Moreover, in the literature, because of the occurrence of hydroxylation reactions, elevated mobility is ascribed to secondary phases in the copper catalyst (A. Prašnikar et al., Ind. Eng. Chem. Res., 2019, 58, 13021). This can further increase the mobility of the copper particles. Moreover, this can result in coverage of the copper particles, which likewise reduces the active surface area. The control of the parameters of temperature and concentration/partial pressures is therefore a crucial challenge in the implementation of direct dimethyl ether synthesis.

In dimethyl ether synthesis, hydrogen and carbon monoxide are reacted by the following reactions:

2H₂+CO⇄MeOH

2MeOH⇄DME+H₂O

CO+H₂O⇄CO₂+H₂

These are equilibrium reactions that result in a different product composition depending on the pressure, temperature and feed composition.

At a ratio of hydrogen to carbon monoxide of 1 without carbon dioxide in the feed gas, the following net reaction is observed:

3H₂+3CO→DME+CO₂

In the case of a stoichiometry value of 2, the following net reaction is dominant:

2H₂+CO→DME+H₂O

Higher alcohols can be formed via a chain extension via insertion of carbon monoxide. This reaction takes place over the copper catalyst (cat.). The insertion proceeds in methyl species that have formed via hydrogenation of adsorbed carbon monoxide or via readsorption of methanol and prior scission of the carbon-oxygen bond. Further hydrogenation of the adsorbed C2 species then leads to formation of acetaldehyde or, after rapid hydrogenation, to formation of ethanol.

2CH₃OH+cat-OH→CH₃O-cat+H₂O

CH₃O-cat+CO→CH₃COO-cat

CH₃COO-cat+H₂→C₂H₅OH

The higher alcohols are dehydrated to the corresponding olefins and may be hydrogenated to paraffins.

Some studies also give support via DFT calculations to the formation of the carbon-carbon bond by insertion of carbon monoxide into adsorbed methyl species that form, for example, from hydrogenation of adsorbed carbon monoxide. The adsorption of methanol, after scission of the carbon-oxygen bond, can also lead to formation of ethanol, again via insertion of carbon monoxide. A higher partial pressure of methanol could therefore also lead to increased formation of by-products (see, for example, Z.-J. Zuo et al., J. Phys. Chem. C 2014, 118, 12890). Further studies on modified copper catalysts additionally also demonstrate the formation of ethanol by coupling of two methanol molecules (J. J. Spifey & A. Egbebi, Chem. Soc. Rev., 2007, 36, 1514).

A further side reaction that occurs in the case of zeolites is the formation of olefins from methanol and/or dimethyl ether. This can already take place at temperatures well below 300° C. and is guided not only by the temperature but also by the dwell time and the catalyst used. This side reaction is followed by the methanol-to-olefins (MTO) and methanol-to-propylene (MTP) mechanisms known from the literature.

In the direct synthesis of dimethyl ether, the following problems in particular have to be solved in the reactor:
Comparatively short service lives of the catalyst owing to elevated aging as a result of prevailing reaction conditions.
By-products in the dimethyl ether product owing to parallel methanol and dimethyl ether synthesis and hence lack of intermediate purification.
Comparatively low productivity in the downstream bed as a result of kinetically controlled conditions and low temperatures.
Temperature control of the reaction for controlled aging.
High costs as a result of catalyst exchange and/or overdesign of the reactor.

One advantage of the direct synthesis of dimethyl ether results from the shift in equilibrium. As a result of separate methanol synthesis in the upstream part of the reactor according to WO 2019/122078 A1, this advantage is exploited only in the downstream part of the reactor. Therefore, lower yields are to be expected in this case. This corresponds to lower productivity in the upstream part of the reactor.

It is an object of the present invention to overcome the disadvantages mentioned in known processes for direct synthesis of dimethyl ether.

### DISCLOSURE OF THE INVENTION

This object is achieved by a process and a plant for preparation of dimethyl ether from synthesis gas according to the respective independent claims. Advantageous configurations and developments are the subject of the dependent claims and of the description that follows.

The present invention proposes a process for synthesis of dimethyl ether, in which a feed gas mixture containing carbon dioxide and/or carbon monoxide and hydrogen is guided through reaction tubes of a cooled shell and tube reactor that are equipped with a first catalyst and a second catalyst (in particular in the form of the aforementioned methanol catalyst and the likewise mentioned dehydration catalyst), in which the carbon dioxide present in the feed gas mixture and/or the carbon monoxide present in the feed gas mixture is at least partly reacted over the first catalyst with the hydrogen present in the feed gas mixture to give methanol, and in which, in the same shell and tube reactor, the methanol is at least partly converted over the second catalyst to dimethyl ether.

In the context of the present invention, it is possible to use shell and tube reactors of a design customary in the art. For further details with regard to corresponding reactors, reference is made to relevant technical literature, for example the article by G. Eigenberger, “Fixed-Bed Reactors”, in Ullmann's Encyclopedia of Industrial Chemistry, volume B 4, 1992, pages 199 to 238. As described in association with figure 4.1 D and E therein, the use of multiple cooling and reaction zones for temperature control in shell and tube reactors is also known per se. The present invention proposes a particularly advantageous zoning, as elucidated hereinafter.

According to the invention, it is envisaged that the reaction tubes are equipped with the first and second catalysts in the form of a structured bed, where the structured bed has two to four regions (also referred to synonymously here as zones) in which the first and second catalysts are provided in physical mixtures with different mixing ratios, where the regions are arranged such that the concentration of the first catalyst (i.e. of the methanol catalyst) increases in a flow direction through the reaction tubes.

The term “structured bed” in the context of the present invention is especially understood to mean a sequence of catalytic regions or zones with different proportions or contents of first and second catalyst (methanol catalyst and dehydration catalyst) in one reactor. Within a region or zone, these proportions are the same or different from one another only because of production-related variances. In the downstream direction, the proportion or content of the first catalyst (methanol catalyst) increases from region to region or zone to zone. What is present within a region or zone of the structured bed is primarily a physical mixture of the first and second catalyst (methanol catalyst and dehydration catalyst). A special form is what is called a bifunctional catalyst. The two catalysts here have been applied to a common support and not physically mixed. The catalyst may have been applied to random packings such as pellets, rings or irregular bodies.

The present invention differs in particular from what are called pre-beds that are known from the prior art, in which a methanol catalyst is diluted with inert material, and which is followed downstream by a mixed bed in the form of a physical mixture of methanol catalyst and dehydration catalyst. Unlike a uniform bed or individual bed, i.e. a uniform physical mixture of methanol catalyst and dehydration catalyst the entire reactor (or, if a pre-bed is used, over the region downstream of the pre-bed), a structured bed enables by reaction regime by different ratios of methanol catalyst and dehydration catalyst.

The first catalyst may, in the context of the present invention, especially be a copper-based catalyst of the type elucidated at the outset, and so reference may be made to the technical literature cited. The second catalyst may likewise be of a kind that is customary in the art. This may especially be an acidic catalyst on zeolites, γ-dialuminum trioxide and aluminosilicate.

In the context of the present invention, the process may especially comprise running the reactor within a pressure range of 30 to 80 bar and/or within a temperature range of 200 to 290° C. and/or with a gas hourly space velocity (GHSV) of 1500 to 4000 h⁻¹.

In a first configuration of the invention, the number of regions that have the bed may in particular be two (shown in table columns 2 in tables 1 and 2 below), where a first of the regions is an upstream region (identified as MIX1 in tables 1 and 2) of 0% to 30% of the length of the reaction tubes and a second of the regions is a downstream region (MIX2) of 25% to 100% of the length of the reaction tubes. (Empty table cells correspond to regions without beds.)

The percentages given here in the table correspond to length ranges of the respective reaction tube. In other words, a section of a reaction tube referred to as A to B % that has a length of X cm extends from a starting position at A×X/100 cm up to an end position at B×x/100 cm. In other words again, the first and second values of the ranges that follow are a first length position of the reaction tube expressed by the percentage and a second length position of the reaction tube expressed by the percentage. Overlapping range figures should be understood such that a range of overlap may be formed by either one or the other section.

In a first configuration of the invention, in the first of the regions a proportion of the first catalyst may be 1 and a proportion of the second catalyst may be X, and in the second of the regions a proportion of the first catalyst may be 1.5 to 4 and a proportion of the second catalyst may be X.

In a second configuration of the invention, the number of regions that have the bed may in particular be three (table columns 3), where a first of the regions (MIX1) is an upstream region of 0% to 30% of the length of the reaction tubes, a second of the regions (MIX2) is a central region of 20% to 70% of the length of the reaction tubes, and a third of the regions (MIX3) is a downstream region of 50% to 100% of the length of the reaction tubes.

In the second configuration of the invention, in the first of the regions a proportion of the first catalyst may be 1 and a proportion of the second catalyst may be X, in the second of the regions a proportion of the first catalyst may be 1.5 to 2 and a proportion of the second catalyst may be X, and in the third of the regions a proportion of the first catalyst may be 3 to 4 and a proportion of the second catalyst may be X.

In a third configuration of the invention, the number of regions that have the bed may in particular be four (table columns 4), where a first of the regions (MIX1) is an upstream region of 0% to 25% of the length of the reaction tubes, a second of the regions (MIX2) is an upstream central region of 20% to 50% of the length of the reaction tubes, a third of the regions (MIX3) is a downstream central region of 50% to 75% of the length of the reaction tubes, and a fourth of the regions (MIX4) is a downstream region of 75% to 100% of the length of the reaction tubes.

In the third configuration of the invention, in the first of the regions a proportion of the first catalyst may be 1 and a proportion of the second catalyst may be X, in the second of the regions a proportion of the first catalyst may be 1.5 to 2 and a proportion of the second catalyst may be X, in the third of the regions a proportion of the first catalyst may be 2.5 to 3 and a proportion of the second catalyst may be X, and in the fourth of the regions a proportion of the first catalyst may be 4 and a proportion of the second catalyst may be X.

In all configurations, X may especially be the same and may be 0.3 to 1.

The configurations elucidated above are illustrated once again hereinafter with reference to the tables already mentioned.

TABLE 1

| | Number of regions | | |
|---|---|---|---|
| | 2 | 3 | 4 |
| | Tube length % | Tube length % | Tube length % |
| MIX1 | 0-30 | 0-30 | 0-25 |
| MIX2 | 25-100 | 20-70 | 20-50 |
| MIX3 | | 50-100 | 50-75 |
| MIX4 | | | 75-100 |

TABLE 2

| | Number of regions | | |
|---|---|---|---|
| | 2 | 3 | 4 |
| MIX1 | 1:X | 1:X | 1:X |
| MIX2 | 1.5-4:X | 1.5-2:X | 1.5-2:X |
| MIX3 | | 3-4:X | 2.5-3:X |
| MIX4 | | | 4:X |

One advantage of the direct synthesis of dimethyl ether results from the increase in equilibrium conversion. As a result of separate methanol synthesis in the upstream part of the reactor according to the prior art, this advantage is exploited only in the downstream part of the reactor. This combination serves for temperature control.

There is lower productivity in the upstream part of the reactor. It has now been found that the advantages of such temperature control can likewise be generated by a suitable mixture of the two different catalysts. As well as the advantage of temperature control, this has the additional effect of increasing the space-time yield. This is especially shown by examples 1 to 3 elucidated below.

In the foremost or upstream portion, as a result of the low proportion of the first catalyst in the mixture, less methanol is formed and hence temperature control is enabled by virtue of the lower exothermicity. At the same time, the high dehydration catalyst content present here ensures enhanced further reaction of the methanol to give dimethyl ether and prevents the buildup of high partial pressures of methanol, which, specifically in the upstream part of the reactor, lead to formation of by-products together with the high partial pressures of carbon monoxide. Even if an equilibrium in the methanol reaction were to be attained, the proportion of the first catalyst here would be lower compared to other embodiments and hence side reactions would evolve to the same degree.

At least one and not more than three further downstream beds should each be executed with respectively higher proportions of the first catalyst. This enables a controlled temperature regime of the reaction. It is thus possible to avoid peak temperatures exceeding 280° C. Ideally, the temperature is kept within a range between 200° C. and 280° C., and particularly between 220° C. and 270° C. This prevents premature aging as a result of sintering effects, for example in the copper component of the first catalyst. Better control of temperature peaks additionally enables avoidance of olefin formation catalyzed by the second catalyst as a side reaction, or of further reactions. The higher proportion of the first catalyst in the downstream portion of the reactor, by virtue of the lower partial pressure of carbon monoxide, is less critical here to by-product formation. This fact is demonstrated in particular by example 4 elucidated below.

The direct synthesis of dimethyl ether is increasingly kinetically controlled with progressive catalyst aging of the copper component of the first catalyst. The proportion of the first catalyst is involved to a crucial degree in the overall conversion of the direct synthesis. A loss of copper activity therefore has the greatest influence on the efficiency of direct synthesis. Higher proportions of the first catalyst toward the end of the bed therefore generally enable better exploitation of the reactor toward the end of the tube and the poorer synthesis gas conversion which is controlled there by low temperatures and partial hydrogen and carbon monoxide pressures. This is shown in particular by a comparison of examples 2 and 3 elucidated below.

It is not untypical for the first catalysts used in the context of the invention that they lose 20% to 30% of their activity within a short time and have only 25% to 40% of the initial activity over the course of their service life. Thus, the downstream beds are advantageously equipped with about 1.5-4 times the proportion of this first catalyst based on the first bed. In spite of the aging, by virtue of the structured bed for an industrial process, it is possible to achieve a higher conversion over a period of time which is now longer, since the downstream beds compensate for or alleviate the loss of conversion in the upstream bed. This enables prolonged exploitation of the bed.

The methanol reaction is promoted by comparison with the dimethyl ether reaction toward the end of the reactor. Any higher selectivities for methanol are not a problem for the inventive solution since methanol is in any case generally recycled into dimethyl ether synthesis processes by virtue of the dimethyl ether equilibrium reaction. The high dehydration catalyst content at the start of the bed thus constitutes an opportunity for conversion of recycled methanol and at the same time prevents competition of the recycled amounts of methanol with the equilibrium reaction of methanol synthesis. This is demonstrated in particular by example 5 elucidated below.

Temperature control is thus possible either via less methanol catalyst in the mixture, i.e. adjustment of the mixing ratio, or by the use of a less active dehydration catalyst with the same methanol content in the mixture.

In order to achieve particularly high methanol formation rates or space-time yields, as well as increasing the content of first catalyst, it is possible in particular to use a second catalyst having particularly high activity in order to very substantially minimize recycled methanol. Further reaction of DME to give olefins should be avoided in this case. The use of the structured bed allows these side reactions to be avoided via control of the reaction temperature. Such elevated by-product formation would lead to coking of the dehydration catalyst and to a rapid decrease in the activity thereof, as known, for example, from methanol-to-olefins processes.

The plant proposed in accordance with the invention for synthesis of dimethyl ether which has a cooled shell and tube reactor having reaction tubes that are equipped with a first catalyst and a second catalyst is set up to guide a feed gas mixture containing carbon dioxide and/or carbon monoxide and hydrogen through the reaction tubes of the shell and tube reactor, to at least partly react the carbon dioxide present in the feed gas mixture and/or the carbon monoxide present in the feed gas mixture with the hydrogen present in the feed gas mixture over the first catalyst to give methanol, and in which, in the same shell and tube reactor, to at least partly convert the methanol over the second catalyst to dimethyl ether.

According to the invention, the reaction tubes are equipped with the first and second catalysts in the form of a structured bed, where the structured bed has two to four regions in which the first and second catalysts are provided in different physical mixtures, where the regions are arranged such that the concentration of the first catalyst increases in a flow direction through the reaction tubes.

A plant of the invention for preparation of dimethyl ether comprises means that train the plant to implement a process as described above. The plant of the invention or the advantageous developments and configurations thereof accordingly profit from the advantages of the respective corresponding process in an analogous manner, and vice versa.

There follows a detailed elucidation of further features and advantages of the invention and of embodiments proceeding from working examples with reference to the appended drawings and with reference to working examples.

### BRIEF DESCRIPTION OF THE FIGURES

FIG. 1 shows a plant in one configuration of the invention in a simplified schematic diagram.

FIG. 2 shows a hydrocarbon selectivity as a function of average reactor temperature in one working example.

In the figures, identical or mutually functionally corresponding elements are referenced by identical reference numerals. Elucidations regarding process steps are correspondingly applicable to apparatus components, and vice versa.

### EMBODIMENT(S) OF THE INVENTION

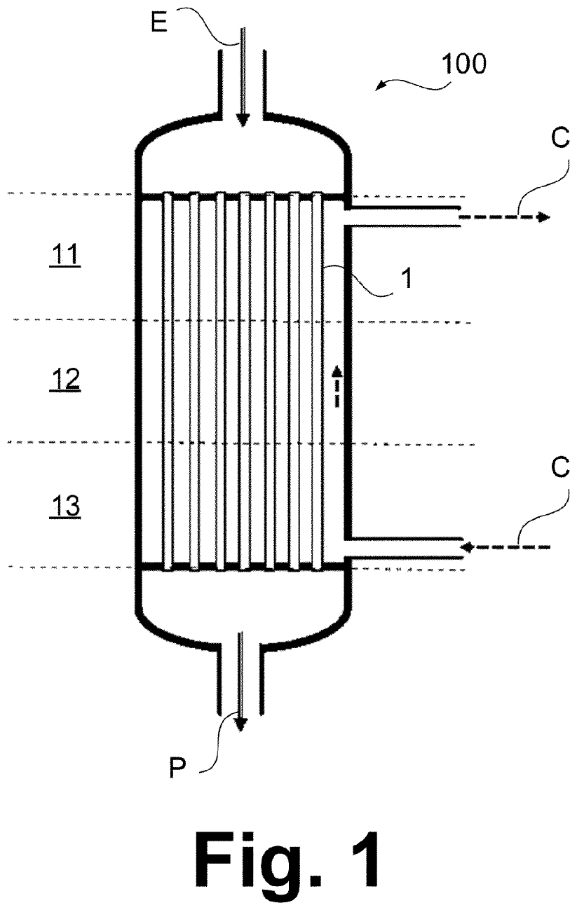

FIG. 1 illustrates a plant of dimethyl ether in greatly simplified form, collectively labeled 100. The plant comprises a shell and tube reactor 10 cooled with a coolant C, having a number of reaction tubes 1. The coolant C is guided through a shell space of the shell and tube reactor 10 in countercurrent to a feed gas mixture E which is guided through the reaction tubes 1, optionally also in the form of several coolant circuits. A product mixture P is withdrawn from the shell and tube reactor 10.

The reaction tubes 1 are equipped with a first catalyst and a second catalyst in the form of a structured bed, where the structured bed in the example described here has three regions 11, 12, 13 in which the first and second catalysts are provided in different physical mixtures, where the regions 11, 12, 13 are arranged such that the concentration of the first catalyst increases in a flow direction through the reaction tubes 1.

### Examples 1 to 3

In the context of examples 1 to 3, a direct synthesis was undertaken by means of a catalyst bed according to the prior art of WO 2019/122078 A1. By comparison, direct syntheses were conducted by means of beds structured in accordance with the invention under similar conditions. The structured beds consisted of two different regions with mixtures of first and second catalyst. The upstream zone was in the range from 0% to 30% of the tube length of the reaction tubes. The first 0-30% always differs from the remaining 25-100%. The parameters are summarized in table 3, with the total content of the first catalyst in percent by weight in the second column of the table, the gas hourly space velocity (GHSV) in the third column of the table, the conversion of carbon monoxide in percent in the fifth column of the table, the space-time yield (STY) of methanol and dimethyl ether as dimethyl ether equivalents in grams of dimethyl ether per kilogram of catalyst per hour in the sixth column of the table, and the selectivity for hydrocarbons in the seventh column of the table.

By virtue of the dimethyl ether equivalents used in the determination of space-time yield, methanol is, in effect, also considered to be dimethyl ether and included in the yield. For this purpose, the following equation may be used:

$$\frac{( {\dot{n}}_{DME} [ \frac{mol}{h} ] + \frac{1}{2} \times {\dot{n}}_{MeOH} [ \frac{mol}{h} ] ) \times {M}_{DME} [ \frac{g}{mol} ]}{{m}_{catalyst} [ kg ]}$$

TABLE 3

| | First cat. | GHSV | Pressure | Conv.CO | STY DME | Sel. for |
|---|---|---|---|---|---|---|
| Ex. | [% by wt.] | [1/h] | [bara] | [%] | [g/(kgₓh)] | HC [%] |
| 1 | 48% | 2000 | 65 | 77.1% | 320 | 2.4% |
| 2 | 46% | 3000 | 65 | 77.% | 480 | 0.7% |
| 3 | 63% | 3000 | 55 | 79.1% | 480 | 1.5% |

In example 1, a diluted methanol pre-bed and a subsequent mixed catalyst bed according to the prior art were used. Example 1 was conducted with a proportion of the first catalyst of 48% by weight, at a pressure of 65 bar (abs.), a GHSV of 2000 h⁻¹, a ratio of hydrogen to carbon monoxide of 1.5, and a stoichiometry value of 1.2. The conversion of carbon monoxide was 77.1%, the selectivity for dimethyl ether 62.3%, the selectivity for methanol 6.8%, the selectivity for carbon dioxide 29.2%, the selectivity for hydrocarbons 2.4%, and the space-time yield of methanol and dimethyl ether as dimethyl ether equivalents in grams of dimethyl ether per kilogram of catalyst per hour 320 g/(kgₓ h).

In example 2, a structured bed consisting of two regions with rising content of the first catalyst was used. Example 2 was conducted with a content of the first catalyst of 46% by weight, at a pressure of 65 bar (abs.), a GHSV of 3000 h⁻¹, a ratio of hydrogen to carbon monoxide of 1.5, and a stoichiometry value of 1.2. The conversion of carbon monoxide was 77.1%, the selectivity for dimethyl ether 62.2%, the selectivity for methanol 6.6%, the selectivity for carbon dioxide 29.3%, the selectivity for hydrocarbons 0.7%, and the space-time yield of methanol and dimethyl ether as defined above 480 g/(kgₓ h).

In example 3, a structured bed consisting of two regions with rising content of the first catalyst was likewise used. Example 3 was conducted with a content of the first catalyst of 68% by weight, at a pressure of 55 bar (abs.), a GHSV of 3000 h⁻¹, a ratio of hydrogen to carbon monoxide of 1.5, and a stoichiometry value of 1.2. The conversion of carbon monoxide was 79.1%, the selectivity for dimethyl ether 60.7%, the selectivity for methanol 9.7%, the selectivity for carbon dioxide 28.9%, the selectivity for hydrocarbons 1.5%, and the space-time yield of methanol and dimethyl ether as defined above 480 g/(kgₓ h).

Comparison between example 1 and example 3 shows the considerably better space-time yield through use of a structured bed. Comparison between example 2 and example 3 shows that the copper content is crucial for the efficiency of the system.

Table 3 shows the temperature profiles for examples 1 to 3 and illustrates that the structured beds, by comparison with the prior art, can both enable temperature control and increase the space-time yield. It also illustrates the potential of accommodating a third zone in the range of 50% to 100% of the tube length, after the temperature peak at about 40% of the tube.

TABLE 3

| | Relative length | | | | | | | | | |
|---|---|---|---|---|---|---|---|---|---|---|
| | [—] | 0.0 | 0.1 | 0.2 | 0.3 | 0.4 | 0.6 | 0.7 | 0.9 | 1.0 |
| T profile of | ° C. | 254 | 277 | 271 | 265 | 277 | 272 | 272 | 268 | 263 |
| example 1 | | | | | | | | | | |
| T profile of | ° C. | 235 | 244 | 262 | 258 | 266 | 263 | 260 | 255 | 252 |
| example 2 | | | | | | | | | | |
| T profile of | ° C. | 236 | 247 | 274 | 262 | 273 | 266 | 258 | 255 | 250 |
| example 3 | | | | | | | | | | |

### Example 4

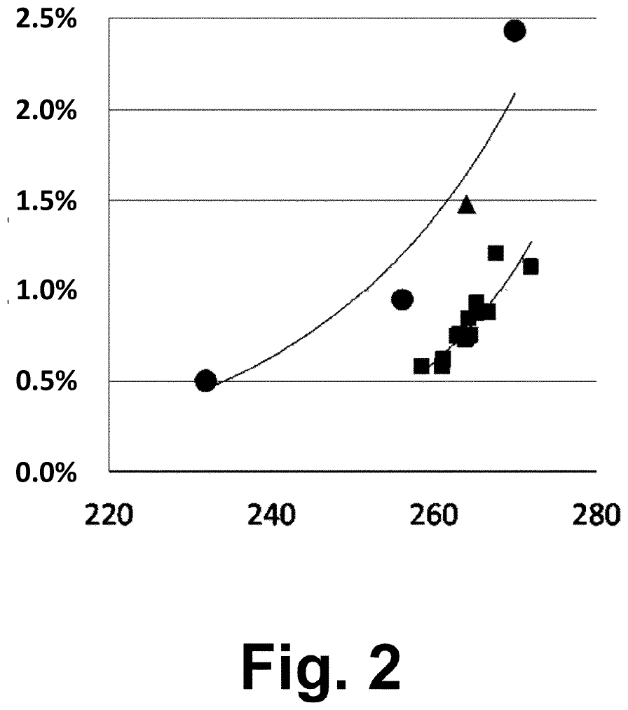

FIG. 2 illustrates hydrocarbon selectivity in percent on the ordinate as a function of the average reactor temperature in ° C. on the abscissa under various reaction conditions, i.e. a ratio of hydrogen and carbon monoxide of 1.5 to 2, a GHSV of 2000 to 4000 h⁻¹, a stoichiometry value of 1 to 3 and a pressure of 50 to 65 bar (abs.) for different bed structures and resulting contents of the first catalyst with different symbols. The results of a comparison between two structured beds (triangles indicate a higher loading and squares a lower loading with the first catalyst) are compared against a bed consisting of a methanol pre-bed and subsequent mixed catalyst bed according to the prior art (circles).

The beds were divided into two different catalytic zones. The upstream zone consisted of about 30% of the active tube length of the cooled tubular reactor used. It was found that, for all beds, selectivity for by-products increases with rising temperature. In addition, it was found that by-product formation in the structured beds increases with rising content of the first catalyst.

Furthermore, it was found that the arrangement according to the prior art has the highest by-product formation even though its content of the first catalyst is similar to the case of the structured bed with the lowest content of the first catalyst, and even though its total copper content is 30% lower in relative terms than that of the structured bed with the highest content of the first catalyst.

This means that, with suitable structuring, higher contents of the first catalyst can be introduced into a bed in order to increase the yield without increasing by-product formation, or even to suppress by-product formation at the same yield.

### Example 5

Example 5 shows the possibility of dosage of methanol at the catalyst bed inlet without seriously disrupting the performance of the bed. The catalyst bed consisted of a 1:1 mixture of first and second catalysts. The results are shown in table 4, where x indicates the respective molar proportions.

Under identical conditions, two reference experiments (table columns 1 and 3, “standard”) were conducted with two different times (time on stream, TOS), namely 193 h and 599 h. Between these references (at 269 h), an experiment was conducted with methanol in the feed gas (“methanol dosage 1”). Comparison between these experiments shows that carbon monoxide conversion is virtually unaffected. This means that methanol recycling has a smaller influence with use of a mixed catalyst system.

TABLE 3

| | | | Methanol | |
|---|---|---|---|---|
| Experiment | [—] | Standard | dosage 1 | Standard |
| TOS | [h] | 193 | 269 | 599 |
| Pressure | bar (abs.) | 60 | 60 | 60 |
| GHSV | GHSV | 3000 | 3000 | 3000 |
| x H2 inlet | mol/mol | 51.0% | 51.4% | 51.3% |
| x CO inlet | mol/mol | 25.2% | 25.5% | 24.3% |
| x CO2 inlet | mol/mol | 4.0% | 4.0% | 4.0% |
| x N2 inlet | mol/mol | 19.8% | 17.0% | 20.0% |
| x O2 inlet | mol/mol | 0.0% | 0.0% | 0.0% |
| x DME inlet | mol/mol | 0.0% | 0.0% | 0.0% |
| x MeOH inlet | mol/mol | 0.0% | 2.2% | 0.0% |
| CO conversion | mol/mol | 71% | 70% | 70% |
| CO2 selectivity | [—] | 25% | 27% | 23% |
| DME selectivity | [—] | 55% | 64% | 51% |
| MeOH selectivity | [—] | 18% | 7% | 24% |
| Selectivity for others | [—] | 0.7% | 0.7% | 0.9% |
| DME yield | [—] | 39% | 45% | 35% |

1.-13. (canceled)

14. A process for synthesis of dimethyl ether, in which a feed gas mixture containing carbon dioxide and/or carbon monoxide and hydrogen is guided through reaction tubes of a cooled shell and tube reactor hat are equipped with a first catalyst and a second catalyst, in which the carbon dioxide present in the feed gas mixture and/or the carbon monoxide present in the feed gas mixture is at least partly reacted over the first catalyst with the hydrogen present in the feed gas mixture to give methanol, and in which, in the same shell and tube reactor, the methanol is at least partly converted over the second catalyst to dimethyl ether, wherein the reaction tubes are equipped with the first and second catalysts in the form of a structured bed, where the structured bed has two to four regions in which the first and second catalysts are provided in different physical mixtures, where the regions are arranged such that the concentration of the first catalyst increases in a flow direction through the reaction tubes.

15. The process according to claim 14, in which the structured bed takes the form of a sequence of catalytic regions each with different proportions of the first and second catalyst, where the proportions of the first and second catalysts within each region are essentially the same.

16. The process according to claim 14, in which the first catalyst is a copper-based catalyst and/or in which the second catalyst is an acidic catalyst, especially based on one or more zeolites, on γ-dialuminum trioxide and/or on aluminosilicate.

17. The process according to claim 14, in which the reactor is run within a pressure range of 30 to 80 bar and/or within a temperature range of 200 to 290° C. and/or with a gas space velocity of 1500 to 4000 h⁻¹.

18. The process according to claim 14, in which the number of regions that have the bed is two, where a first of the regions is an upstream region of 0% to 30% of the length of the reaction tubes, and a second of the regions is a downstream region of 25% to 100% of the length of the reaction tubes.

19. The process according to claim 18, in which, in the first of the regions, a proportion of the first catalyst is 1 and a proportion of the second catalyst is X, and in which, in the second of the regions, a proportion of the first catalyst is 1.5 to 4 and a proportion of the second catalyst is X.

20. The process according to claim 14, in which the number of regions that have the bed is three, where a first of the regions is an upstream region of 0% to 30% of the length of the reaction tubes, a second of the regions is a central region of 20% to 70% of the length of the reaction tubes, and a third of the regions is a downstream region of 50% to 100% of the length of the reaction tubes.

21. The process according to claim 20, in which, in the first of the regions, a proportion of the first catalyst is 1 and a proportion of the second catalyst is X, in which, in the second of the regions, a proportion of the first catalyst is 1.5 to 2 and a proportion of the second catalyst is X, and in which, in the third of the regions, a proportion of the first catalyst is 3 to 4 and a proportion of the second catalyst is X.

22. The process according to claim 14, in which the number of regions that have the bed is four, where a first of the regions is an upstream region of 0% to 25% of the length of the reaction tubes, a second of the regions is an upstream central region of 20% to 50% of the length of the reaction tubes, a third of the regions is a downstream central region of 50% to 75% of the length of the reaction tubes, and a fourth of the regions is a downstream region of 75% to 100% of the length of the reaction tubes.

23. The process according to claim 22, in which, in the first of the regions, a proportion of the first catalyst is 1 and a proportion of the second catalyst is X, in which, in the second of the regions, a proportion of the first catalyst is 1.5 to 2 and a proportion of the second catalyst is X, in which, in the third of the regions, a proportion of the first catalyst is 2.5 to 3 and a proportion of the second catalyst is X, and in which, in the fourth of the regions, a proportion of the first catalyst is 4 and a proportion of the second catalyst is X.

24. The process according to claim 19, in which X is 0.3 to 1.

25. A plant for synthesis of dimethyl ether which has a cooled shell and tube reactor having reaction tubes that are equipped with a first catalyst and a second catalyst, and which is set up to guide a feed gas mixture containing carbon dioxide and/or carbon monoxide and hydrogen through the reaction tubes of the shell and tube reactor, to at least partly react the carbon dioxide present in the feed gas mixture and/or the carbon monoxide present in the feed gas mixture with the hydrogen present in the feed gas mixture over the first catalyst to give methanol, and in which, in the same shell and tube reactor, to at least partly convert the methanol over the second catalyst to dimethyl ether, wherein the reaction tubes are equipped with the first and second catalysts in the form of a structured bed, where the structured bed has two to four regions in which the first and second catalysts are provided in different physical mixtures, where the regions are arranged such that the concentration of the first catalyst increases in a flow direction through the reaction tubes.

26. The plant (100) according to claim 25, set up to implement a process for synthesis of dimethyl ether, in which a feed gas mixture containing carbon dioxide and/or carbon monoxide and hydrogen is guided through reaction tubes of a cooled shell and tube reactor hat are equipped with a first catalyst and a second catalyst, in which the carbon dioxide present in the feed gas mixture and/or the carbon monoxide present in the feed gas mixture is at least partly reacted over the first catalyst with the hydrogen present in the feed gas mixture to give methanol, and in which, in the same shell and tube reactor, the methanol is at least partly converted over the second catalyst to dimethyl ether, wherein the reaction tubes are equipped with the first and second catalysts in the form of a structured bed, where the structured bed has two to four regions in which the first and second catalysts are provided in different physical mixtures, where the regions are arranged such that the concentration of the first catalyst increases in a flow direction through the reaction tubes.
